(19) United States
(12) Patent Application Publication
(10) Pub. No.: US 2023/0285060 A1
(43) Pub. Date: Sep. 14, 2023

(54) FRACTURE AND NONUNION RIB IMPLANTS

(71) Applicant: Zimmer Biomet CMF and Thoracic, LLC, Jacksonville, FL (US)

(72) Inventors: Darren DeMedici, Middletown, NJ (US); James Fewell, Neptune Beach, FL (US); Jose Ramos, Jersey City, NJ (US); Jack Sopotnick, Jacksonville, FL (US); Max Holland Billard, Jacksonville, FL (US)

(21) Appl. No.: 18/108,900

(22) Filed: Feb. 13, 2023

Related U.S. Application Data
(60) Provisional application No. 63/319,559, filed on Mar. 14, 2022.

(51) Int. Cl. A61B 17/80

(52) U.S. Cl. CPC A61B 17/8076; A61B 17/8009

(57) ABSTRACT

A rib implant system can include a first implant, a second implant, and a reduction member. The first implant can be securable to a first rib portion of a rib. The second implant can be securable to a second rib portion of the rib. The reduction member can be connected to the first implant and the second implant, and the reduction member can be operable to move the second implant and the second rib portion toward the first implant and the first rib portion.

### CLAIM OF PRIORITY

This application claims the benefit of U.S. Provisional Patent Application Ser. No. 63/319,559, filed on Mar. 14, 2022, the benefit of priority of which is claimed hereby, and which is incorporated by reference herein in its entirety.

### BACKGROUND

The present invention relates generally to apparatus and systems for rib implants. For patients with various rib injuries, surgeons sometimes install a plate to secure fractured rib portions to each other. In some cases, multiple fractures can occur. In these cases, additional hardware may be used to help regain anatomical integrity between the rib segments and to protect organs directly behind the ribcage. In some examples, surgeons use donor tissue to fasten the rib segments to each other.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of a rib implant coupled to a rib cage of a patient.

FIG. 2A illustrates an isometric view of a rib implant assembly secured to a rib.

FIG. 2B illustrates an isometric view of a rib implant assembly secured to a rib.

FIG. 3A illustrates an isometric view of a rib implant assembly secured to a rib.

FIG. 3B illustrates an isometric view of a rib implant assembly secured to a rib.

FIG. 4 illustrates an isometric view of a rib implant assembly secured to a rib.

FIG. 5 illustrates an isometric view of a portion of a rib implant assembly.

FIG. 6 illustrates an isometric view of a portion of a rib implant assembly.

FIG. 7 illustrates an isometric view of a rib implant assembly.

FIG. 8A illustrates a side view of a portion of a rib implant assembly.

FIG. 8B illustrates an isometric view of a portion of a rib implant assembly.

FIG. 9A illustrates an isometric view of a portion of a rib implant assembly.

FIG. 9B illustrates an isometric view of a portion of a rib implant assembly.

FIG. 10A illustrates an isometric view of a portion of a rib implant assembly secured to a rib.

FIG. 10B illustrates an isometric view of a rib implant assembly secured to a rib.

FIG. 10C illustrates an isometric view of a rib implant assembly secured to a rib.

FIG. 10D illustrates an isometric view of a rib implant assembly secured to a rib.

FIG. 11A illustrates an isometric view of a rib implant assembly.

FIG. 11B illustrates an isometric view of a portion of a rib implant assembly.

FIG. 11C illustrates an isometric view of a portion of a rib implant assembly.

FIG. 11D illustrates an isometric view of a portion of a rib implant assembly.

FIG. 11E illustrates an isometric view of a portion of a rib implant assembly.

FIG. 11F illustrates a schematic view of a portion of a rib implant assembly.

FIG. 12 illustrates an isometric view of a rib implant assembly.

FIG. 13 illustrates an isometric view of a rib implant assembly secured to a rib.

FIG. 14A illustrates an isometric view of a portion of a rib implant assembly.

FIG. 14B illustrates an isometric view of a portion of a rib implant assembly.

FIG. 15 illustrates an isometric view of a rib implant assembly.

FIG. 16 illustrates an isometric view of a rib implant assembly.

FIG. 17 illustrates an isometric view of a rib implant assembly.

### DETAILED DESCRIPTION

In some cases of a broken rib, surgeons use plates and screws to bridge the fracture and to secure rib segments to each other. These types of fastening systems may also be difficult to use in cases of multiple fractures, such as where reduction of floating rip portions require approximation and reduction to a position near their normal anatomical location.

This disclosure can help to address these issues by using a rib implant assembly securable to each of the fractured pieces. The assembly can include multiple implants each securable to the rib portions. Once the implants are secured to the portions, a reduction feature that is connected to, or integral to, the implant assembly can be operated to approximate and reduce the rib portions. Once the rib portions are reduced, the implants can be further secured to each other or other rib portions to secure the implants and rib portions to each other to repair the fracture(s).

The above discussion is intended to provide an overview of subject matter of the present patent application. It is not intended to provide an exclusive or exhaustive explanation of the invention. The description below is included to provide further information about the present patent application.

As used herein, the terms “proximal” and “distal” should be given their generally understood anatomical interpretation. The term “proximal” refers to a direction generally toward the torso of a patient or base or handle of a tool, and “distal” refers to the opposite direction of proximal, i.e., away from the torso of a patient or toward the working end of the tool.

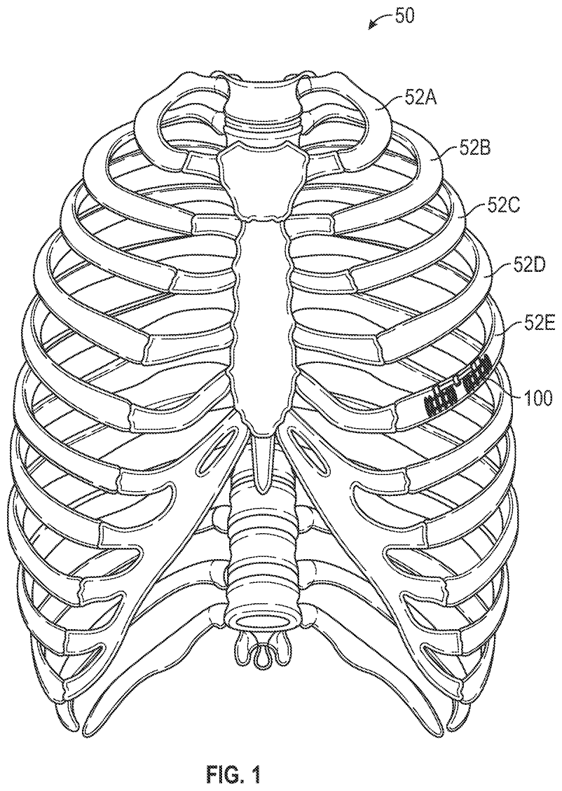

FIG. 1 shows an isometric view of a rib implant coupled to a rib cage of a patient. FIG. 1 shows the rib cage 50 including ribs 52A-52E and an implant assembly 100. In some examples, the rib cage 50 can be a rib cage of a human, such as a patient, where the rib 52E can be a fractured rib of the rib cage 50. In this example, the rib implant assembly 100 can be secured to adjacent ends of the rib 52E, which may be fractured and/or resected. Once secured to adjacent ends of the rib 52E, rib implant 100 can provide a bridge between rib portions. Further details of the rib implant assembly 100 are discussed below with respect to the figures below.

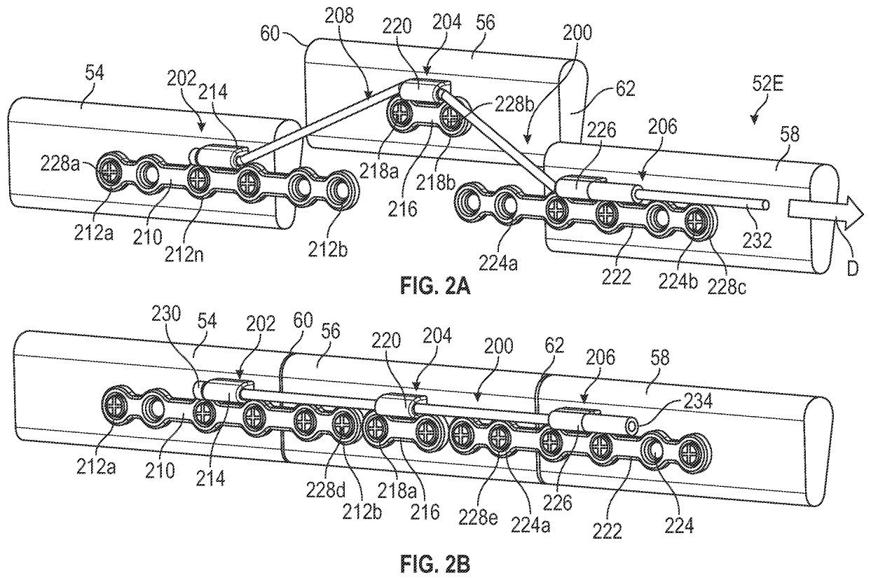

FIG. 2A illustrates an isometric view of a rib implant assembly 200 secured to a rib 52. FIG. 2B illustrates an isometric view of the rib implant assembly 200 secured to the rib 52. FIGS. 2A and 2B are discussed together below. The assembly 200 can include a first implant 202, a second implant 204, a third implant 206, and a reduction member 208. FIGS. 2A and 2B also show rib portions 54, 56, and 58, and fractures 60 and 62.

The first implant 202 can include a first body 210, first bores 212 (e.g., 212a-212n), and a first reduction feature 214. The second implant 204 can include a second body 216, second bores 218 (e.g., 218a-218n), and a second reduction feature 220. The third implant 206 can include a third body 222, third bores 224 (e.g., 224a-224n), and a third reduction feature 226. The rib implant assembly 200 can also include one or more fasteners 228 (e.g., 228a-228n).

Each body of the first implant 202, the second implant 204, and the third implant 206 can be a rigid or semi-rigid member made of a single piece (or multiple pieces in some examples) and can be made of biocompatible materials such as one or more of stainless steels, cobalt chromium, titanium, or the like. In some examples, portions of the bodies can be made of porous or semi-porous materials configured to promote bone ingrowth to enhance fixation (such as through osseointegration) of the bodies (210, 216, or 222) to respective portions of the rib 52E. One porous material that can be used is OsseoTi™ porous metal from Zimmer Biomet™ (Warsaw, Ind.). OsseoTi can be made of Ti6Al4V and can have a porous structure that generally mimics a porous structure of human cancellous bone. Also, the porous material can be Trabecular Metal™, also from Zimmer Biomet. Such a material may be formed from a reticulated vitreous carbon foam substrate that can be infiltrated and coated with a biocompatible metal, such as tantalum, such as using a chemical vapor deposition (“CVD”) process in the manner disclosed in detail in U.S. Pat. No. 5,282,861. Also, the porous material can be Regenerex®, also from Zimmer Biomet. In other examples, other porous materials can be used.

The bodies (210, 216, or 222) can each define one or more of the bores 212, 218, and 224, respectively, extending therethrough. Each bore can be configured to receive a fastener therethrough and can be configured to retain a portion (such as a head) of the fastener therein, such as to secure each of the bodies to a bone, such as a portion of the rib 52E. The fasteners 228 can each be one or more of screws, bolts, rivets, or the like. Though FIG. 2B shows 12 of the fasteners 228, fewer fasteners, such as 3, 4, 5, 6, 7, 8, 9, 10, or the like can be used in some examples. In other examples, more fasteners can be used, such as 15, 20, 25, 35, or the like. As shown in FIG. 2B, optionally, some of the bores may not be used to receive fasteners.

The reduction member 208 can be a cord, rope, tether, wire, or the like, that is an elongate and optionally flexible member. The reduction member 208 can be fixed or connected to the first reduction feature 214 at a first end 230 of the reduction member 208, such as via a knot or other retention mechanism. A second end 232 can be loose in a first configuration (shown in FIG. 2A) and can be secured using a clamp 234 in a second configuration (shown in FIG. 2B). The reduction member 208 can also extend through the second reduction feature 220 and the third reduction feature 226.

In operation of some examples, when it is desired to repair a fractured rib, such as the rib 52E, the rib implant assembly 200 can be used to approximate, reduce, and secure the rib portions 54, 56, and 58. For example, the first body 210 can be secured to the rib portions 54 using fasteners 228 secured to the rib portions 54 through some of the first bores 212, such as to secure the first implant 202 to the rib portions 54. For example, the fastener 228a can be secured to the rib portions 54 through the bore 212a and other bores of the first implant 202 can extend from the rib portions 54, such as the bore 212h Similarly, the third body 222 can be secured to the rib portions 58 using fasteners 228 secured to the rib portions 58 through some of the third bores 224, such as to secure the first implant 202 to the rib portions 54. For example, the fastener 228c can be secured to the rib portions 58 through the bore 224b and other bores of the first implant 202 can extend from the rib portions 54, such as the bore 224a. The second body 216 can be secured to the rib portion 56 by securing fasteners to the rib portion 56 through the bores 218a and 218b. Also, the reduction member 208 can be passed, or positioned, through the first reduction feature 214, the second reduction feature 220, and the third reduction feature 226.

Once the implants are secured to the rib portions and the reduction member 208 is secured to the implants, the reduction member 208 can be operated, such as by, pulling on the reduction member 208 in a direction D to tighten the reduction member 208 through the retention features. Such operation or action of the reduction member 208 can approximate the separated rib portion 56 (shown in FIG. 2A) with the rib portions 54 and the rib portions 58. Further operation of the reduction member 208 can also reduce the rib portion 56 with respect to the rib portions 54 and the rib portions 58. Following approximation and reduction, as shown in FIG. 2B, additional fasteners can be secured to the implants to secure the assembly to the rib portions. For example, the bore 212b can receive the fastener 228d to secure the first implant 202 to the rib portion 56 and the bore 224a can receive the fastener 228e to secure the third implant 206 to the rib portion 56 thereby by securing, respectively, the first implant 202 to the rib portion 56 and the second implant 204 to the rib portion 56 to secure the rib implant assembly 200 to all three portions of the rib 52E and to fixate the portions to each other.

Optionally, following securing of the first implant 202, the second implant 204, and the third implant 206 to the rib portions, the reduction member 208 can be removed from the rib implant assembly 200 before the opening is closed. Alternatively, the clamp 234 can be used to secure the reduction member 208 to the third reduction feature 226 of the third implant 206, such as after the reduction member 208 is shortened to remove excess material, Knots, ties, clips, or other devices can be used instead of the clamp 234 to secure the reduction member 208.

In this way, the rib implant assembly 200 can be used to approximate, reduce, and secure two or more fractured portions of a rib (or other bone) to each other to repair the fractures 60 and 52. Optionally, the first implant 202, second implant 204, and third implant 206 can be secured to a posterior surface of the rib to help reduce palpability of the rib implant assembly 200,

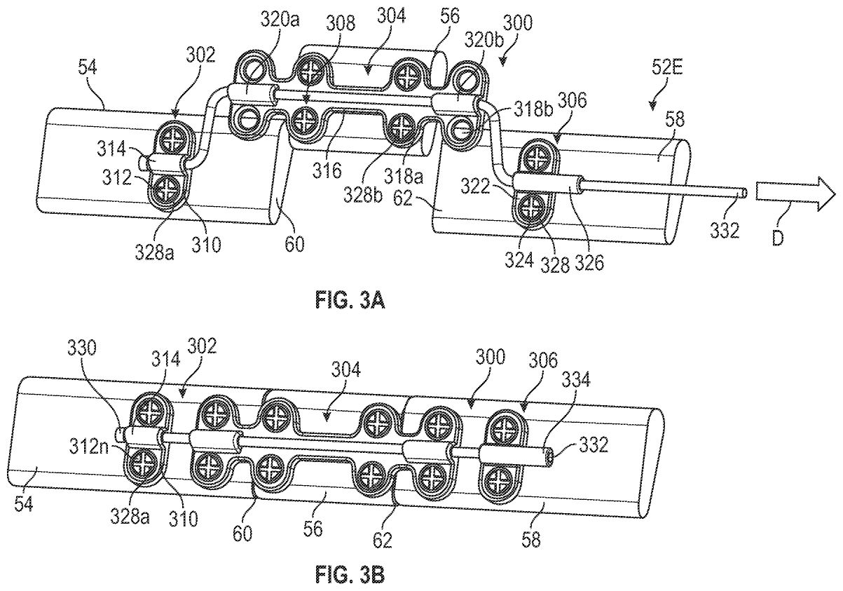

FIG. 3A illustrates an isometric view of a rib implant assembly 300 secured to a rib 52E. FIG. 3B illustrates an isometric view of the rib implant assembly 300 secured to the rib 52E. FIGS. 3A and 313 are discussed together below. The rib implant assembly 300 can be similar to the rib implant assembly 200 discussed above. The rib implant assembly 300 can be different in that the rib implant assembly 300 can include a second implant that is relatively larger and connects to adjacent rib portions. Any of the implant assemblies discussed above or below can be modified to include such features.

The rib implant assembly 300 can include a first implant 302, a second implant 304, a third implant 306, and a reduction member 308. The first implant 302 can include a first body 310, first bores 312, and a first reduction feature 314. The second implant 304 can include a second body 316, second bores 318, and a second reduction feature 320. The third implant 304 can include a third body 322, third bores 324, and a third reduction feature 326. Also, the reduction member 308 can include a first end 330 and a second end 332 that can be secured via a clamp 334.

The first implant 302 and the third implant 306 can include only two bores each, helping to reduce a size of the first implant 302 and the third implant 306 while the second implant 304 can include many bores and multiple second reduction features 320. This configuration can allow the first implant 302 and the third implant 306 to be completely secured to the rib portions 54 and the rib portions 58, respectively, prior to approximation and reduction and only the second implant 304 is secured to the rib portions 54 and rib portions 58 following reduction of the rib portions. This embodiment also provides four retention features (two on the second implant 304), which can help improve approximation and reduction of the second implant 304 and the rib portion 56 in line with the rib portions 54 and the rib portions 58 prior to fixation of the second implant 304 to the rib portions 54 and the rib portions 58.

Each of the implants of the rib implant assembly 300 can also include bores located on either side of the retention features (and therefore on either side of the retention member 308), which can help reduce movement of the implants due to torque during reduction and can help protect the bone (e.g., the rib 52E) during approximation and reduction. The rib implant assembly 300 can thereby include relatively fewer bores (such as compared to the rib implant assembly 200) while still providing good fixation to the rib 52E.

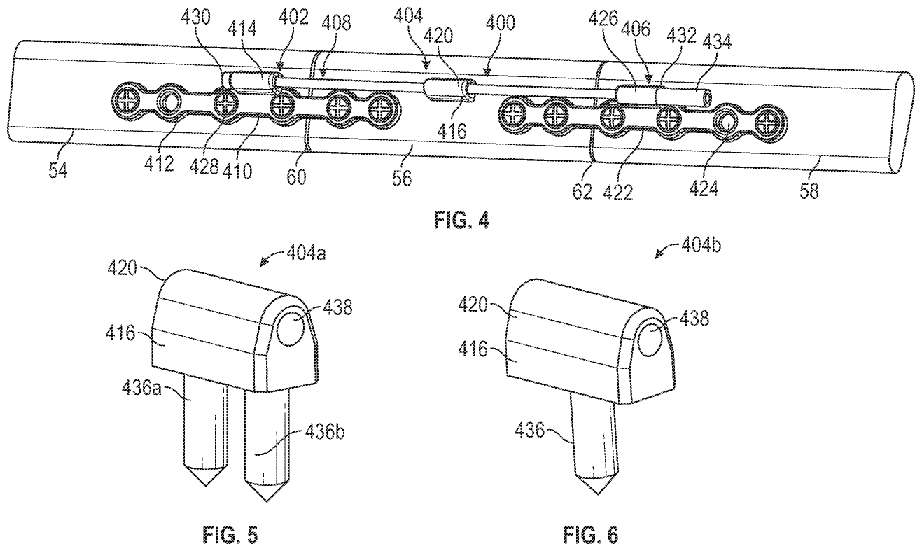

FIG. 4 illustrates an isometric view of a rib implant assembly 400 secured to a rib. The rib implant assembly 400 can be similar to the rib implant assemblies discussed above. The rib implant assembly 400 can be different in that the rib implant assembly 400 can include a second implant that is secured to the rib portion using only bone spikes. Any of the implant assemblies discussed above or below can be modified to include such features.

The rib implant assembly 400 can include a first implant 402, a second implant 404, a third implant 406, and a reduction member 408. The first implant 402 can include a first body 410, first bores 412, and a first reduction feature 414. The second implant 404 can include a second body 416 and a second reduction feature 420. The third implant 406 can include a third body 422, third bores 424, and a third reduction feature 426. Also, the reduction member 408 can include a first end 430 and a second end 432 that can be secured via a clamp 434.

The rib implant assembly 400 can be connected to the rib portions 54, 56, and 58 similarly to the rib implant assemblies 200-300 discussed above except that the second implant 404 includes no fastener bores and can therefore be secured to the rib portion 56 via one or more bone spikes. Use of a bone spike can help save time during installation of the rib implant assembly 400 during a surgical procedure. Once the rib portions 54, 56, and 58 are approximated and reduced, the first implant 402 and the third implant 406 can be secured to the rib portion 56 (such as using fasteners 428 through the bores 412 and 424) to secure the rib portions 54, 56, and 58 to the rib implant assembly 400. By being relatively small and simple, the second implant 404 can help lower the cost of the rib implant assembly 400. Also, the second implant 404 can be more easily removed following securing of the first implant 402 and the third implant 406 to the rib portion 56 allowing for a reduced number of components permanently (or indefinitely) implanted. The reduction member 408 can optionally be removed following securing the first implant 402 and the third implant 406 to the rib portion 56 and removal of the second implant 404.

FIG. 5 illustrates an isometric view of a second implant 404a that can be similar to the second implant 404 of the rib implant assembly 400. FIG. 5 shows additional details of the second implant 404a, For example, FIG. 5 shows that the second implant 404a can include the second body 416 and the second reduction feature 420, where the second reduction feature 420 is connected to (or defined by) the second body 416. For example, the second reduction feature 420 can define a bore 438 extending therethrough. The bore 438 can be sized and shaped to receive the reduction member 408 therein or therethrough and can be configured to retain the reduction member 408 therein. The bore 438 can be configured to limit movement of the reduction member 408 with respect to the second implant 404 in non-axial directions.

FIG. 5 also shows bone spikes 436a and 436b, which can be bone spikes extending from the second body 416 away from the second reduction feature 420 (and optionally perpendicular to the bore 438. The bone spikes 436a and 436b can each be configured to insert into bone in a friction or interference engagement such as to secure the second implant 404a to the bone. Though two bone spikes are shown, 3, 4, 5, 6, or the like bone spikes can be used.

FIG. 6 illustrates an isometric view of a second implant 404b that can be similar to the second implant 404 of the rib implant assembly 400. The second implant 404b can also be similar to the second implant 404a except that the second implant 404b can include only a single bone spike 436. Use of a single spike can help reduce cost of the 404h and can reduce a number of bores in the bone, Reducing a number of bores in the bone can be beneficial to healing when it is desired to remove the second implant 404b, such as following securing the first implant 402 and the third implant 406 to the rib portion 56, or such as following removal of the entire rib implant assembly 400.

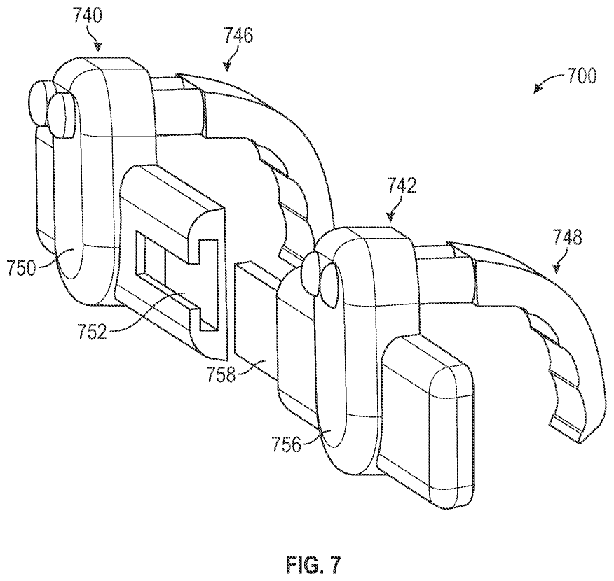

FIG. 7 illustrates an isometric view of a rib implant assembly 700. The rib implant assembly 700 can be similar to the assemblies discussed above. The rib implant assembly 700 can differ in that it can use clamps instead of fasteners (such as screws or spikes). Any of the rib implant assemblies discussed above or below can be modified to includes such features.

The rib implant assembly 700 can include a first implant 740 and a second implant 742, The first implant 740 can include a hook 746 and the second implant 742 can include a hook 748. The first implant 740 can include a first body 750 including a reduction member 752 and the second implant 742 can include a second body 756 including a reduction member 758. The reduction member 752 can optionally be a slot extending into at least a portion of the first body 750 and the reduction member 758 can be a bar or projection extending from the second body 756. The bar 758 can be insertable into the slot 752 to, together, define a reduction member or reduction assembly of the rib implant assembly 700. In use, the bar 758 can be translatable within the slot 752 to guide movement of the first implant 740 with respect to the second implant 742.

The hook 746 can be secured to the first body 750 and can be configured to engage one or more portions of a rib to secure the first implant 740 to the rib. Similarly, the hook 748 can be secured to the first body 750 and can be configured to engage one or more portions of a rib to secure the first implant 740 to the rib. Further details of the rib implant assembly 700 and operation of the rib implant assembly 700 are discussed below.

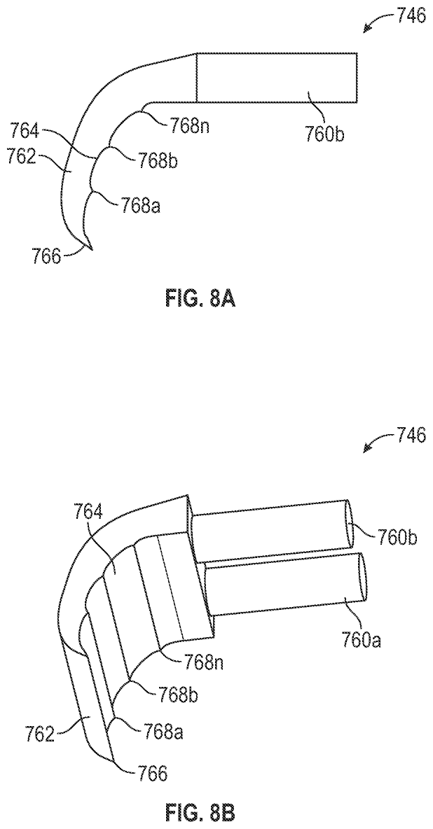

FIG. 8A illustrates a side view of the hook 746 of the rib implant assembly 700. FIG. 8B illustrates an isometric view of the hook 746 of the rib implant assembly 700. FIGS. 8A and 8B are discussed together below. Though FIGS. 8A and 8B discuss the hook 746, all of the features discussed with regard to the hook 746 can be included in the hook 748.

The hook 746 can include rods 760a and 760b extending from an engagement portion 762. The rods 760 can each be cylindrical but can be other shapes in other examples, such as a rectangular prism, or the like. Optionally, the hook 746 can include one rod or can include more than two rods, such as 3, 4, 5, or the like.

The engagement portion 762 can have a relatively curved shape defining an engagement surface 764, which can be a radially inner portion of the engagement portion 762 configured to engage bone. The engagement surface 764 can be sized and shaped to be complementary to a rib to increase clamping effectiveness of the 746. The engagement portion 762 can also define a tip 766 at an end of the engagement portion 762 that can be relatively sharp and configured to surround and retain at least a portion of a bone or rib. The tip 766 can optionally be configured to penetrate the bone or rib.

Also, the engagement portion 762 can include ribs or projections 768 (e.g., 768a-768n) extending radially inward from the engagement surface 764. The projections 768 can be relatively sharp and can be configured to engage bone (e.g., to at least partially penetrate the bone, such as a rib) to limit movement of the hook 746 with respect to the bone once the hook 746 is engaged with the bone or rib. Though three of the projections 768 are shown, the engagement portion 762 can include 1, 2, 4, 5, 6, 7, 8, 9, 10, 15, 20, or the like projections.

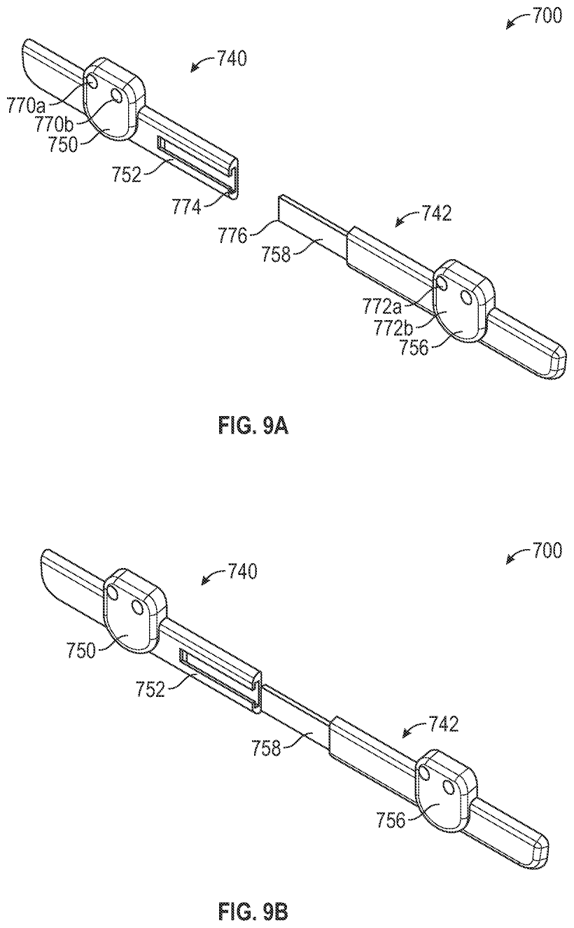

FIG. 9A illustrates an isometric view of the implant 740 and FIG. 9B illustrates an isometric view of the implant 742. FIGS. 9A and 9B are discussed together below.

As discussed above, the first implant 740 can include the first body 750 and the reduction member 752 extending from the first body 750. Also, the second implant 742 can include the second body 756 and a bar 758 extending from the second body 756. FIG. 9A shows that the first body 750 can include bores 770a and 770b configured to receive the rods 760a and 760b, respectively, of the hook 746 therein or therethrough. Similarly, the second body 756 can include bores 772a and 772b configured to receive rods 760a and 760b, respectively, of the hook 748 therein or therethrough.

FIG. 9A also shows that the reduction member 752 can define a slot having an open end 774 configured to receive an end 776 of the bar 758 therein for insertion of the bar 758 into the reduction member 752 such as to secure the first body 750 to the reduction member 752 and to allow for guided movement of the first implant 740 with respect to the second implant 742 such as for approximation or reduction of fractured rib portions, as discussed in further detail below.

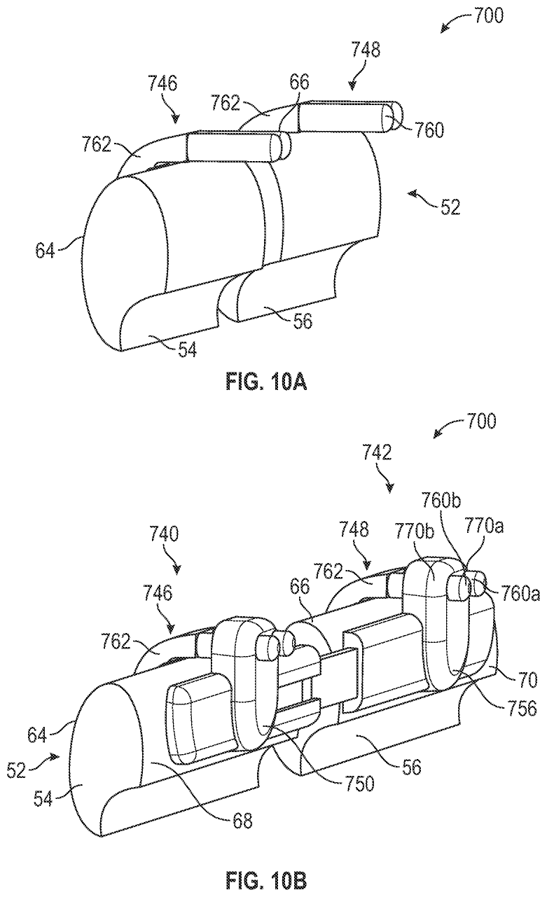

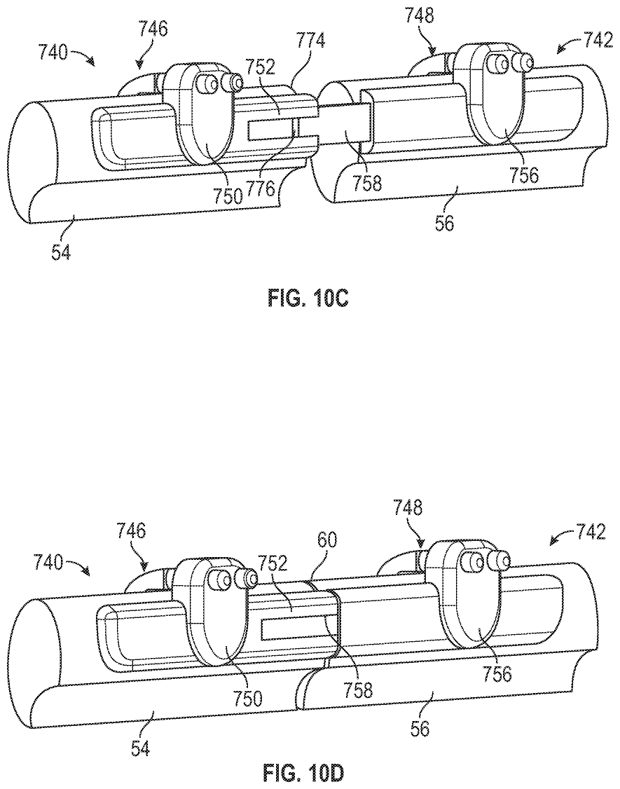

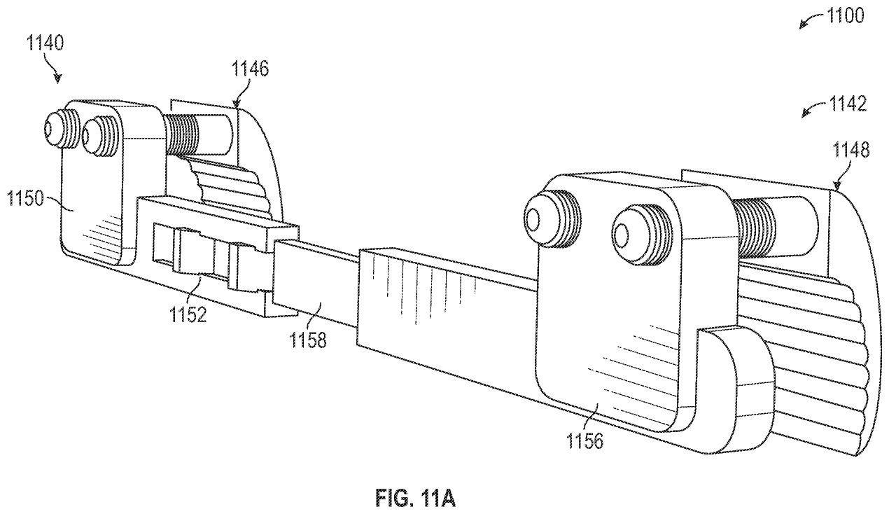

FIGS. 10A-10D each illustrate a step of a process using isometric views of the rib implant assembly 700 and a rib 52. For example, FIG. 10A shows how the hook 746 can be positioned to engage a first side or surface 64 of the rib portion 54 and the hook 748 can be positioned to engage a first side or surface 66 of the rib portion 56. For example, the engagement surface 764, the tip 766, and the projections 768 of the hook 746 can engage the surface 64 of the rib portions 54. When the hook 746 is engaged with the surface 64, the rods 760 can extend over (e.g., above or superior to) or around a portion of the rib portions 54 and 56. Optionally, the hook 746 and the hook 748 can engage a posterior portion of the rib 52, such as to allow an anterior approach to installation to be used for a fracture repair on an anterior portion of the rib cage (as shown in FIG. 1). Conversely, the hook 746 and the hook 748 can engage an anterior portion of the rib 52 to locate the first body 750 and the second body 756 on a posterior side (or internal side) of the rib to help limit palpability of the first body 750 and the second body 756.

FIG. 10B shows that the first body 750 can be engaged with a surface 68 of the rib portion 54, opposing the surface 64. Similarly, the second body 756 can be engaged with a surface 70 of the rib portion 56, opposing the surface 66. During this step, the rods of the hooks can be secured to the bodies. For example, the rods 760a and rods 760b can be inserted into the bores 770a and 770b, respectively, such as to secure the hooks 746 and 748 to the bodies 750 and 756, respectively. When the hook 746 is secured to the first body 750 the rib portion 54 can be clamped or secured to the first implant 740 and when the hook 748 is secured to the second body 756, the rib portion 56 can be clamped or secured to the second implant 742. Optionally, the rods 760 can be secured to the bodies 750 and 756 using a fastener, such as a nut, to maintain the clamping force on the rib portions 54 and 56,

FIG. 100 shows that when the first implant 740 and the second implant 742 are secured to the rib portion 54 and the rib portion 56, respectively, the end 776 of the bar 758 can be inserted into the open end 774 of the reduction member 752, such as to approximate the rib portion 54 with the rib portion 56 prior to reduction. In this orientation or position, the hook 746 and the hook 748 together with the first body 750 and the second body 756, respectively, can maintain their position or location with respect to the rib portions 54 and 56 through the clamped interface.

FIG. 10D shows how the bar 758 can be translated within the reduction member 752 to allow the second implant 742 and the rib portion 56 to move with respect to the first implant 740 and the rib portion 54 to reduce the rib portions 54 and the rib portion 56 while being guided by the bar 758 and the channel 752. The bar 758 and the channel 752 can help to guide proper reduction to align the rib portions 54 and 56 to close or reduce the fractures 60 between the rib portions 54 and 56. In this way, the rib implant assembly 700 can be used to repair a rib (or other bone) fracture without the use of penetrating fasteners; however, penetrating fasteners can optionally be incorporated into the rib implant assembly 700 for additional securing of the first implant 740 or the second implant 742.

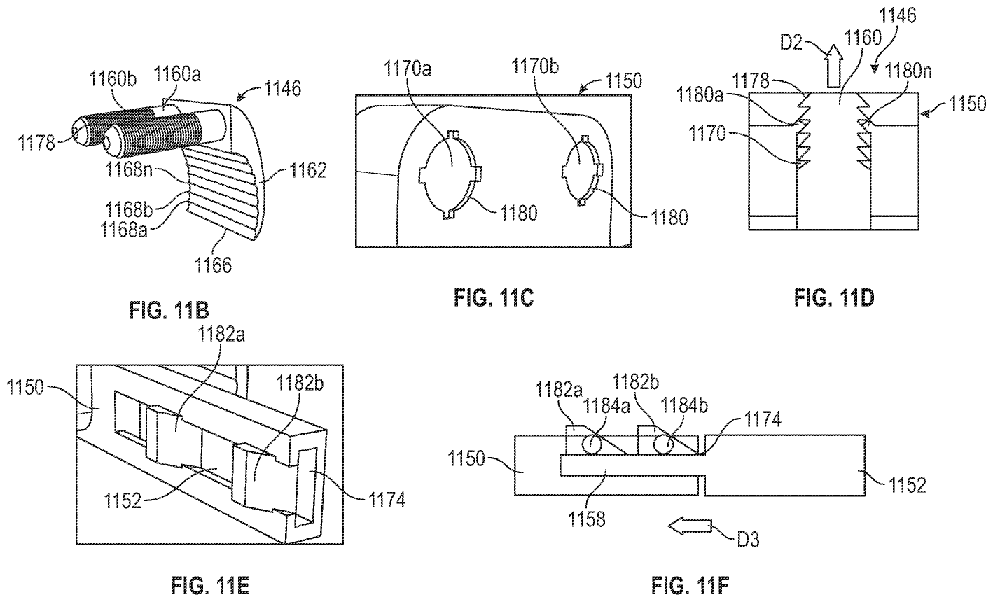

FIG. 11A illustrates an isometric view of a rib implant assembly 1100. The rib implant assembly 1100 can be similar to the assemblies discussed above. The rib implant assembly 1100 can differ in that it can use ratcheting interfaces to secure components of the assembly. Any of the rib implant assemblies discussed above or below can be modified to include such features.

The rib implant assembly 100 can include a first implant 1140 and a second implant 1142. The first implant 1140 can include a hook 1146 and the second implant 1142 can include a hook 1148. As discussed in further detail below, the hooks 1146 and 1148 can include rods having ratcheting features for securing the hooks 1146 and 1148 to the bodies 1150 and 1156, respectively.

The first implant 1140 can include a first body 1150 including a reduction member 1152 and the second implant 1142 can include a second body 1156 including a reduction member 1158, The reduction member 1152 can optionally be a slot extending into at least a portion of the first body 1150 and the reduction member 1158 can be a bar or projection extending from the second body 1156. The bar 1158 can be insertable into the slot 1152 to, together, define a reduction member or reduction assembly. The bar 1158 can be translatable within the slot 1152 to guide movement of the first implant 1140 with respect to the second implant 1150, The reduction member 1152 can also include ratcheting features for controlling movement of the reduction member or bar 1158 with respect to the slot 1152, such as during approximation and reduction of rib portions, as discussed in further detail below.

FIG. 11B illustrates an isometric view of the hook 1146 of the rib implant assembly 1100. FIG. 11C illustrates an isometric view the body 1150 the rib implant assembly 1100. FIG. 11D illustrates an isometric view of the hook 1146 and the body 1150 of the rib implant assembly 1100. FIGS. 11B-11D are discussed together below.

FIG. 11B shows that the hook 1146 can include an engagement portion 1162 that can have a relatively curved shape defining an engagement surface 1164, which can be a radially inner portion of the engagement portion 1162 configured to engage bone. The engagement portion 1162 can also define a tip 1166 at an end of the engagement portion 1162 that can be relatively sharp and configured to surround and retain a portion of a bone or rib and which can be configured to penetrate and retain the bone or rib. Also, the engagement portion 1162 can include ribs or projections 1168 (e.g., 1168a-1168n) extending radially inward from the engagement surface 1164. The projections 1168 can relatively sharp and configured to engage bone to limit movement of the hook 1146 with respect to the bone once engaged. The engagement portion 1162 can include 1, 2, 3, 4, 5, 6, 7, 9, 10, or the like projections.

FIG. 11B also shows that the hook 1146 can include a rod 1160a and a rod 1160b (rods 1160) connected to the engagement portion 1162. The rods 1160 can each be cylindrical but can be other shapes in other examples, such as a rectangular prism, or the like. Optionally, the hook 1146 can include one rod or more than two rods, such as 3, 4, 5, or the like. As shown in FIGS. 11B and 11D, each rod 1160 can include a plurality of teeth 1178 on an outer surface of the rods, where the teeth 1178 can be spaced axially along a length of the rods 1160.

FIG. 11C shows that the body 1150 can include bores 1170a and 1170b that can extend at least partially through the body 1150. The body 1156 can include similar bores. The bores 1170a and 1170b can be sized and shaped to receive the rods 1160a and 1160b therein or therethrough, such as to secure the hook 1146 to the body 1150. The body 1150 or the bores 1170 can optionally include pawls 1180a-1180n. The pawls 1180 can be projections, tabs, extensions, or the like that can extend radially inward from the body 1150 into the bores 1170. FIG. 11D shows that the teeth 1160a-1160n can extend radially outward and can be angled for unidirectional axial movement through the bores 1170 and past the pawls 1180 such as to form a ratcheting interface between the rods 1160 and the body 1150.

In operation of some examples, the body 1150 can be engaged with a first side of rib portion, and the hook 1146 (or the engagement portion 1162 thereof) can be engaged with a second side of the rib (opposite the first side). The rods 1160 can be inserted into the bores 1170 (before or during engagement of the body 1150 and the hook 1146) to secure the hook 1146 to the body 1150. As shown in FIG. 11D, during insertion of the rods 1160 into the bores 1170, the teeth 1178 can deflect when the rods 1160 moves in the direction D2 to allow movement of the rods 1160 further into the bore 1170. Following deflection of each tooth a flat side of the tooth can engage the pawls 1180 to limit movement of the rod 1160 in a direction opposite direction D2, which can help limit separation of the hook 1146 from the body 1150 and can help maintain compressive forces on the rib to help keep the implant 1140 secured to the rib.

FIG. 11E illustrates an isometric view of the body 1150 of the first implant 1140 and FIG. 11F illustrates a schematic view of the first implant 1140 and the second implant 1142. FIGS. 11E and 11F are discussed together below.

FIGS. 11E and 11F show that the reduction member 1152 can include a channel opening 1174 and can define a slot therein configured to receive the reduction member 1158 of the reduction member 1152. FIGS. 11E and 11F also show that the reduction member 1152 can include wedges 1182a and 1182b and balls 1184a and 1184b located within the wedges, respectively. The balls 1184 can move within their respective wedges 1182 to allow movement of the bar 1158 in the direction D3, but the balls 1184 can lock up with the wedges 1182, respectively, to limit movement of the bar 1158 in a direction opposite direction D3, such as to allow reduction of the first implant 1140 and the second implant 1142 and the rib portions to which they connect, and to limit separation of the first implant 1140 and the second implant 1142.

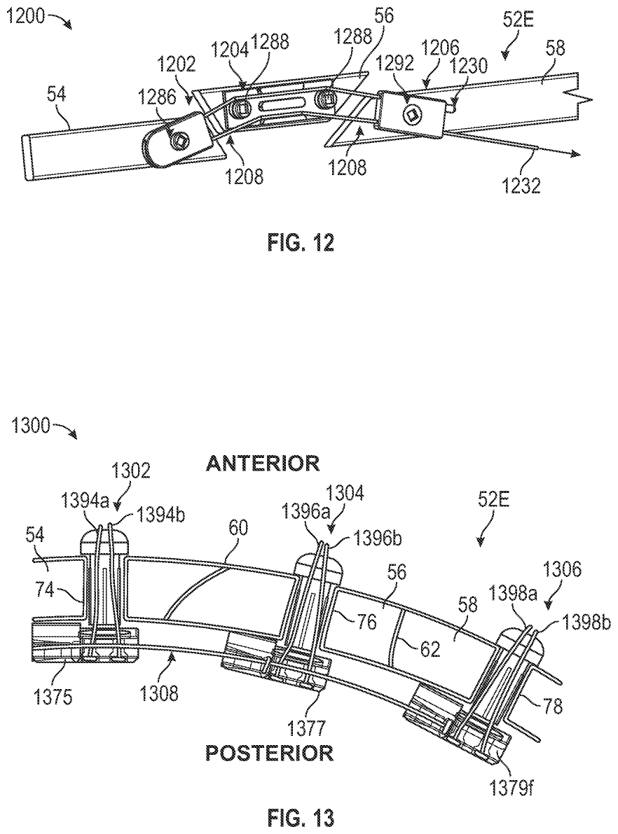

FIG. 12 illustrates an isometric view of a rib implant assembly 1200. The rib implant assembly 1200 can be similar to the assemblies discussed above. The rib implant assembly 1200 can differ in that it can use actuators therein to reduce the implants and rib portions. Any of the rib implant assemblies discussed above or below can be modified to includes such features. FIG. 12 also shows rib 52E and rib portions 54, 56, and 58.

The rib implant assembly 1200 can include a first implant 1202, a second implant 1204, a third implant 1206, and a reduction member 1208. The reduction member 1208 can extend through the third implant 1206 where a first end 1230 can be secured to the third implant 1206. The 1208 can extend through the second implant 1204 and the first implant 1202 and can wrap around the first implant 1202 and return to the third implant 1206 where a second end 1232 can be free for adjusting a tension of the 1208.

The first implant 1202 can include a first actuator 1286, the second implant 1204 can include a second actuator 1288 and third actuator 11290, and the third implant 1206 can include a fourth actuator 1292. The actuators can be operated to move the reduction member 1208 through the implants, such as to approximate and reduce the rib portions 54, 56, and 58, Following reduction, the second end 1232 can be trimmed and secured to the third implant 1206. The rib implant assembly 1200 can optionally include fewer or more implants and fewer or more actuators, such as to optimize reduction of the fractures of the rib 52E.

FIG. 13 illustrates an isometric view of a rib implant assembly 1300 secured to a rib 52E. The rib implant assembly 1300 can be similar to the assemblies discussed above. The rib implant assembly 1300 can differ in that it can use implants insertable into a bore and connectable to a posterior connector. Any of the rib implant assemblies discussed above or below can be modified to includes such features. FIG. 13 also shows rib 52E and rib portions 54, 56, and 58, which can be separated by fractures 60 and 62 and can include bores 74, 76, and 78, respectively.

The rib implant assembly 1300 can include a first implant 1302, a second implant 1304, a third implant 1306, and a reduction member 1308. The first implant 1302 can be insertable through the bore 74, the second implant 1304 can be insertable through the bore 74, and the third implant 1306 can be insertable through the bore 78. The rib implant assembly 1300 can also include a first set of tethers 1394a and 1394b, a second set of tethers 1396a and 1396b, and a third set of tethers 1398a and 1398b.

In operation, the first implant 1302 can be inserted into the bore 74, the second implant 1304 can be inserted into the bore bores 76, and the third implant 1306 can be inserted into the bore bores 78, such as anteriorly to posteriorly through their respective bores, until a head of each implant engages an anterior surface of its respective rib. The tethers then each be connected to posterior connectors 1375, 1377, and 1379, For example, the posterior connector 1375 can be inserted to engage a posterior portion of the rib portions 54, and the tethers 1394a and 1394b can be inserted through the first implant 1302 and the bore 74 to secure the first implant 1302 to the posterior connectors 1375. The second implant 1304 and the third implant 1306 can be similarly connected. Once the posterior connectors 1375, 1377, and 1379 are secured to their respective implants 1302, 1304, and 1306, the reduction member 1308 can be connected to the posterior connectors 1375, 1377, and 1379 (or can be previously connected thereto). Once the reduction member 1308 is connected to the posterior connectors 1375, 1377, and 1379, the reduction member 1308 can be operated to move the first implant 1302, the second implant 1304, and the third implant 1306 to reduce and approximate the rib portions 54, the rib portion 56, and the rib portions 58 to close the fractures 60 and the fractures 62. The reduction member 1308 can be tightened as necessary to secure the implants and the rib portions to each other.

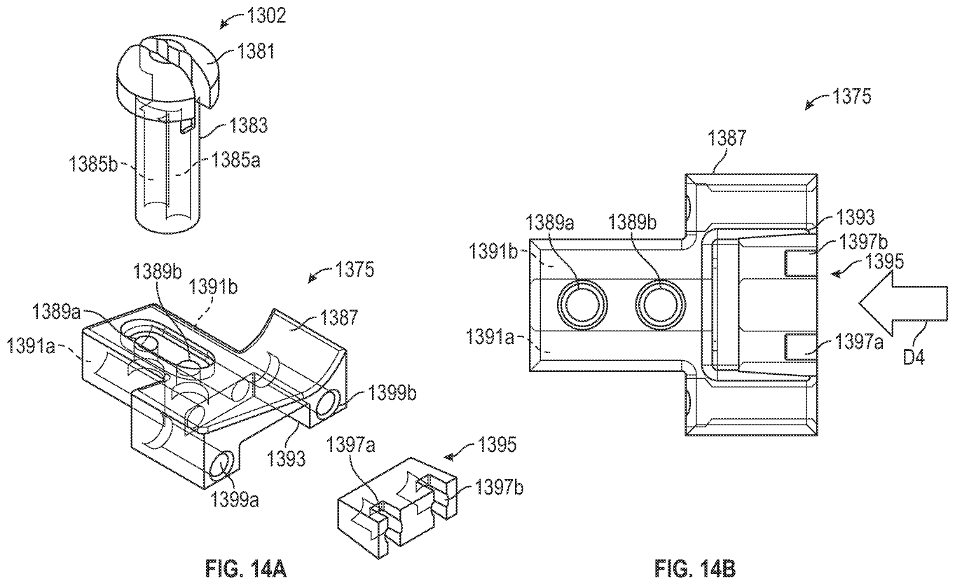

FIG. 14A illustrates an isometric exploded view of the first implant 1302 and the posterior connectors 1375 of the rib implant assembly 1300. FIG. 14B illustrates an isometric top view of the posterior connectors 1375 of the rib implant assembly 1300. FIGS. 14A and 14B are discussed together below. The first implant 1302 and the posterior connectors 1375 of the rib implant assembly 1300 can be consistent with the rib implant assembly 1300 and the posterior connectors 1375 of FIG. 13. FIGS. 14A and 14B show additional details of the first implant 1302 and the posterior connectors 1375.

For example, FIG. 14A shows that the first implant 1302 can include a head 1381 and a shank 1383. The shank 1383 can extend from the head 1381 and can have a diameter or width smaller than that of the head 1381 such that the shank 1383 can extend into or through a bore of the rib 52 and the head 1381 can engage the rib 52 to limit extension of the first implant 1302 therein or therethrough. The shank 1383 can be cylindrical, such as to be complementary to the bore, but can have other shapes, such as a rectangular prism, hexagonal prism, octagonal prism, or the like. The first implant 1302 can also include bores 1385a and 1385b that can extend through the shank 1383 and the head 1381. The bores 1385a and 1385b can be sized and shaped to receive the tethers 1394a and 1394b, respectively. The 1381 can optionally be sized or configured to retain an end portion of the tethers 1394a and 1394b when the tethers 1394a and 1394b are within the bores 1385a and 1385b. The head 1381 can optionally include a slot to allow the 1381 to be crimped or crushed to secure the tethers 1394a and 1394b within the bores 1385.

FIGS. 14A and 14B also show that the posterior connector 1375 can include a body 1387 defining bores 1389a and 1389b extending at least partially therethrough. The bores 1389a and 1389b can be configured to receive the tethers 1394a and 1394b therein or therethrough to connect the first implant 1302 to the posterior connectors 1375, such as when the first implant 1302 is located within a bore of the rib 52. The bores 1389a and 1389b can optionally be countersunk or counterbored to retain an end portion of the tethers 1394a and 1394b.

FIGS. 14A and 14B also show that the body 1387 can include bores 1391a and 1391b that can extend at least partially through the body 1387, such as transverse to the bores 1389. The bores 1391 can intersect or extend into to an insert slot 1393, which can be sized and shaped to receive an insert 1395 therein. The insert 1395 can include retainers 1397a and 1397b. The retainers 1397a and 1397b can be aligned with the bores 1391a and 1391b, respectively.

The posterior connectors 1375 can also optionally include bores 1399a and 1399b extending and least partially through the body 1387, such as parallel to the bores 1391. The bores 1399 can be configured to each retain one or more tethers. For example, the tethers can connect to the posterior connectors 1375 via the bores 1399 or the bores 1391.

In operation, the shank 1383 can be inserted into a bore of the rib 52E and tethers can be secured to the bores 1389 and the bores 1385 to secure the first implant 1302 to the posterior connectors 1375. The head 1381 can then be crushed to secure the tethers within the bores 1385 and the tethers can be knotted or otherwise secured to the body 1387. The body 1387 can be pressed to an opposing side of the rib from the first implant 1302 and can be sized and shaped to conform to the rib (e.g., curved). Adjacent posterior connectors 1375 can be connected to each other using tethers connected via the bores 1391 or the bores 1399. The 1395 can be inserted in direction D4 into the insert slot 1393 of each body 1387 to secure the tethers connecting the posterior connectors 1375. The insert 1395 can be shaped to have an interference fit with the insert slot 1393 such that sides of the insert 1395 interfere with tethers within the bores 1399. Insertion of the insert 1395 into the insert slot 1393 can also cause sides of the insert 1395 to deflect inward to crush or clamp tethers within the retainers 1397, helping to secure the tethers to the body 1387 and the posterior connectors 1375 to each other.

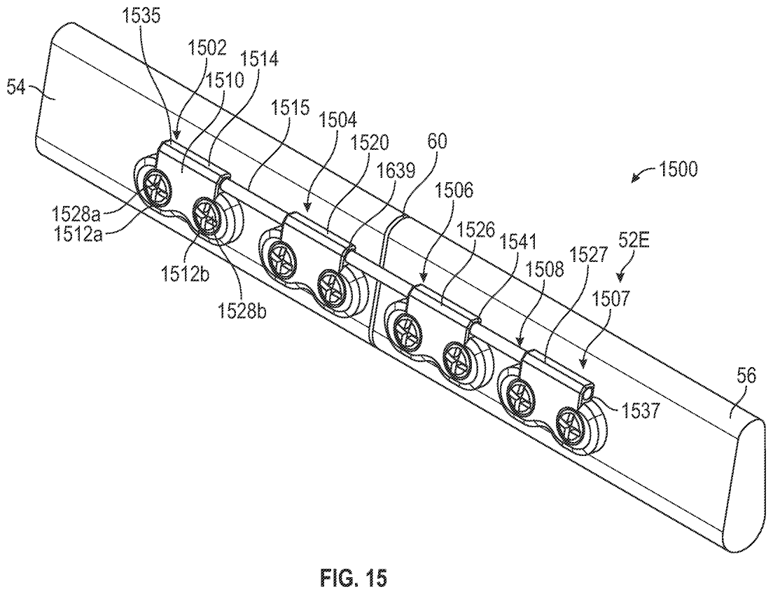

FIG. 15 illustrates an isometric view of a rib implant assembly 1500. The rib implant assembly 1500 can be similar to the assemblies discussed above. The rib implant assembly 1500 can differ in that it can include a flexible tether secured to outer implants and can include inner implants free to move along the tether. Any of the rib implant assemblies discussed above or below can be modified to includes such features. FIG. 15 also shows rib 52E including rib portions 54 and 56 and a fracture 60.

The rib implant assembly 1500 can include a first implant 1502, a second implant 1504, a third implant 1506, a fourth implant 1507, and a tether member 1508. Each implant can include a body and reduction features. For example, the first implant 1502 can include a body 1510 and a retention feature 1514 connected to the body 1510. The second implant 1504 can include a bore 1515 therein or therethrough.

Each body of the first implant 1502, the second implant 1504, the third implant 1506, and the fourth implant 1507 can be a rigid or semi-rigid member made of a single piece (or multiple pieces in some examples) and can be made of biocompatible materials such as one or more of stainless steels, cobalt chromium, titanium, or the like. In some examples, portions of the bodies can be made of porous or semi-porous materials configured to promote bone ingrowth to enhance fixation (such as through osseointegration) of the bodies (e.g., 1510) to respective portions of the rib 52E. One porous material that can be used is OsseoTi™ porous metal from Zimmer Biomet™ (Warsaw, Ind.) OsseoTi can be made of Ti6Al4V and can have a porous structure that generally mimics a porous structure of human cancellous bone. Also, the porous material can be Trabecular Metal™, also from Zimmer Biomet. Such a material may be formed from a reticulated vitreous carbon foam substrate that can be infiltrated and coated with a biocompatible metal, such as tantalum, such as using a chemical vapor deposition (“CND”) process in the manner disclosed in detail in U.S. Pat. No. 5,282,861. Also, the porous material can be Regenerex®, also from Zimmer Biomet. In other examples, other porous materials can be used.

The bodies can each define fastener bores extending therethrough. For example, the body 1510 can define bores 1512a and 1512b. Each bore can be configured to receive a fastener therethrough and can be configured to retain a portion (such as a head) of the fastener therein, such as to secure each of the bodies to a bone, such as a portion of the rib 52E. The fasteners 1528a and 1528b can each be one or more of screws, bolts, rivets, or the like. Though FIG. 15 shows 8 of the fasteners 1528, fewer fasteners or more fasteners, such as 3, 4, 5, 6, 7, 8, 9, 10, 12, 15, 20, or the like can be used in some examples. Optionally, some of the bores may not be used to receive fasteners.

The tether 1508 can be a cord, rope, tether, wire, or the like, that is an elongate and optionally flexible or malleable member. Optionally, the tether 1508 can be a semi-rigid and malleable rod providing some flex for shaping of the tether 1508 to match contouring of the rib 52E and allowing positioning of the implants to match such contouring. The reduction member 1508 can be fixed or connected to the first retention feature 1514 at a first end 1535 of the reduction member 1508 and can be connected to a fourth retention feature 1527 at a second end 1537, such as via a weld, crip, or using one or more fasteners.

Optionally, bores 1539 and 1541 of retention features 1520 and 1526, respectively, of the second implant 1504 and the third implant 1506, respectively, can receive the tether 1508 therethrough without being fixed to the tether 1508. That is, the second implant 1504 and the third implant 1506 can be free to move along the tether 1508 where such movement is guided or limited by engagement with the retention features 1520 and 1526, respectively. This can allow the second implant 1504 and the third implant 1506 to move with respect to the first implant 1502, the second implant 1504, or the tether 1508 such as to be located or positioned as desired during a procedure. For example, the second implant 1504 can be be positioned on the rib portions 54 (on a first side of the fracture 60) and the third implant 1506 can be positioned on the rib portions 58 (on a second side of the fracture 60), In this way, the implants 1502-1507 and the tether 1508 can be used to conform to a shape or curvature of a rib (e.g., the rib 52E) and can be located with respect to the fracture 60 as desired, allowing the rib implant assembly 1500 to be used in a variety of applications including floating rib applications where one implant is attached to a floating portion for approximation, reduction, and fixation of the three or more rib portions.

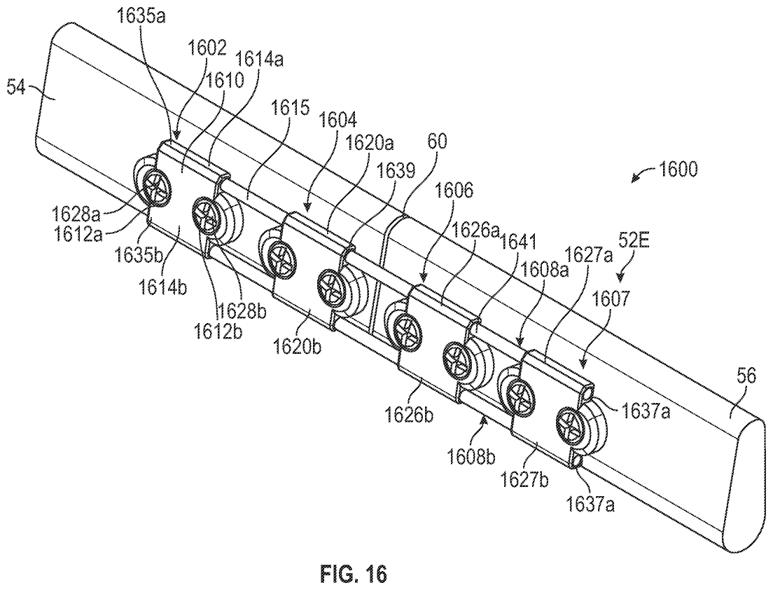

FIG. 16 illustrates an isometric view of a rib implant assembly 1600. The rib implant assembly 1600 can be similar to the assemblies discussed above. The rib implant assembly 1600 can differ in that it can include multiple tethers for securing the plates to each other. Any of the rib implant assemblies discussed above or below can be modified to includes such features. FIG. 16 also shows rib 52E including rib portions 54 and 56 and a fracture 60.

The rib implant assembly 1600 can be similar to the rib implant assembly 1500; the rib implant assembly 1600 can differ in that each implant can include second retention features. Accordingly, like components can be indicated by like numerals. More specifically, each of a first implant 1602, a second implant 1604, a third implant 1606, and a fourth implant 1607 can include top and bottom retention features. For example, the first implant 1602 can include a top retention feature 1614a and a bottom retention feature 1614b. The second implant 1604 can include a top retention feature 1620a and a bottom retention feature 1620b. The third implant 1606 can include a top retention feature 1626a and a bottom retention feature 1626b. The fourth implant 1607 can include a top retention feature 1627a and a bottom retention feature 1627b. The rib implant assembly 1600 can also include a first (or top) tether 1608a and a second (or bottom) tether 1608b.

The top retention features 1614a, 1620a, 1626a, and 1627a can receive the tether 1608a therein or therethrough and the bottom retention features 1614b, 1620b, 1626b, and 1627b can receive the tether 1608b therein or therethrough. The tethers 1608a and 1608b can be secured to the implants 1602 and 1607 (such as at first ends 1635a and 1635b and second ends 1637a and 1637b. In this way, the tethers 1608a and 1608b can still allow for flexibility and positioning of the second implant 1604 and the third implant 1606 along the tethers 1608, but can help to provide additional rigidity or stability of the rib implant assembly 1600 before or after securing of the rib implant assembly 1600 to the rib 52E.

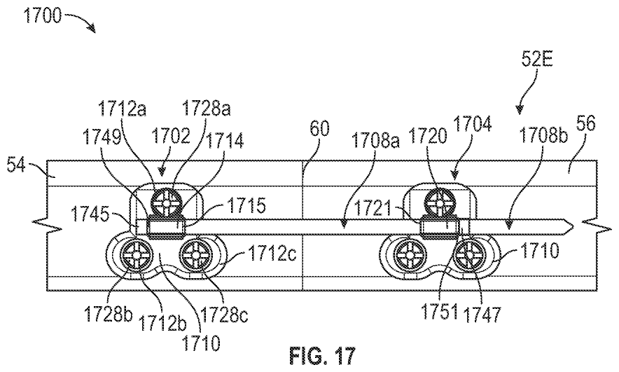

FIG. 17 illustrates an isometric view of a rib implant assembly 1700. The rib implant assembly 1700 can be similar to the assemblies discussed above. The rib implant assembly 1700 can differ in that it can include a locking tether secured to the implants. Any of the rib implant assemblies discussed above or below can be modified to includes such features. FIG. 17 also shows rib 52E including rib portions 54 and 56 and a fracture 60.

The rib implant assembly 1700 can include a first implant 1702 and a second implant 1704, The first implant 1702 can include a first body 1710 defining bores 1712a-1712c, and can include a first retention feature 1714 (defining a bore or slot or passage 1715). Similarly, the second implant 1704 can include a second body 1716 and a second retention feature 1720 (defining a bore or slot or passage 1721). The rib implant assembly 1700 can also include a tether 1708 which can include a first portion 1708a, a second portion 1708b, a head 1745, and a lock 1747. The first portion 1708a can be two portions secured together, or can be a single portion with a sliding lock. The first retention feature 1714 and the second retention feature 1720 can receive and retain the tether 1708 therein or therethrough, such as through the slot 1715 and the slot 1721, such as to align the second implant 1704 with the first implant 1702 and to align the second rib portion 56 with the first rib portion 54.

In operation of some examples, the first implant 1702 can be secured to the rib portions 54 using fasteners and the second implant 1704 can be secured to the rib portion 56 using fasteners. Optionally, the tether 1708 can then be used to align, approximate, or reduce the rib portions 54 and the rib portion 56. Once the rib portions 54 and the rib portion 56 are aligned, such as when it is desired to secure the tether 1708 (such as following securing the first implant 1702 and the second implant 1704 via screws or fasteners), the head 1745 can be engaged with an end 1749 of the first retention feature 1714. The lock 1747 can then be operated to secure the first portion 1708a to the second portion 1708b and to engage the lock 1747 with an end. 1751 of the 1720, in this way, the lock 1747 can be used to fix the relative positions of the first implant 1702, the second implant 1704, the rib portion 54, and the rib portion 56, or can at least limit relative motion thereof.

### NOTES AND EXAMPLES

The following, non-limiting examples, detail certain aspects of the present subject matter to solve the challenges and provide the benefits discussed herein, among others.

Example 1 is a rib implant system comprising: a first implant securable to a first rib portion of a rib; a second implant securable to a second rib portion of the rib; and a reduction member connected to the first implant and the second implant, the reduction member operable to move the second implant and the second rib portion toward the first implant and the first rib portion.

In Example 2, the subject matter of Example 1 optionally includes wherein the first implant includes a first fastener bore to receive a first fastener therethrough to secure the first implant to the first rib portion.

In Example 3, the subject matter of Example 2 optionally includes wherein the second implant includes a second fastener bore to receive a second fastener therethrough to secure the second implant to the second rib portion, and includes an overlap fastener bore to receive a third fastener therethrough to secure the second implant to the first rib portion.

In Example 4, the subject matter of any one or more of Examples 1-3 optionally include a third implant securable to a third portion of the rib, the third implant including a reduction passage extending through at least a portion of the third implant, the reduction passage to receive at least a portion of the reduction member therethrough to align the third implant with the first implant and the second implant and to align the third rib portion with the second rib portion and the first rib portion when the reduction member is adjusted from a first configuration to a second configuration.

In Example 5, the subject matter of Example 4 optionally includes wherein the first implant includes a first fastener bore to receive a first fastener therethrough to secure the first implant to the first rib portion, wherein the second implant includes a second fastener bore to receive a second fastener therethrough to secure the second implant to the second rib portion, and wherein the third implant includes a third fastener bore to receive a fastener therethrough to secure the third implant to the third rib portion.

In Example 6, the subject matter of any one or more of Examples 4-5 optionally include wherein the third implant includes a bone spike engageable with the third rib portion to secure the third implant to the third rib portion.

In Example 7, the subject matter of any one or more of Examples 4-6 optionally include wherein the reduction member is a tether secured to the first implant and engageable with the second implant, the tether extendable through the third implant, such that operation of the tether aligns the third implant with the first implant and the second implant to align the third rib portion with the first rib portion and the second rib portion.

In Example 8, the subject matter of Example 7 optionally includes wherein the second implant includes a second reduction passage extending through at least a portion of the second implant, the second reduction passage to receive at least a portion of the tether therethrough to align the second implant with the first implant and the third implant and to align the second rib portion with the third rib portion and the first rib portion when the reduction member is operated.

In Example 9, the subject matter of any one or more of Examples 7-8 optionally include wherein the third implant includes a second reduction passage extending through at least a portion of the third implant, the second reduction passage to receive at least a portion of the tether therethrough to align the third implant with the first implant and the second implant and to align the third rib portion with the first rib portion and the second rib portion when the reduction member is operated.

In Example 10, the subject matter of any one or more of Examples 1-9 optionally include wherein the first implant includes a first body engageable with a first side of the first rib portion and a first hook engageable with a second side of the first rib portion to secure the first implant to the first rib portion.

In Example 11, the subject matter of Example 10 optionally includes wherein the second implant includes a second body engageable with a first side of the second rib portion and a second hook engageable with a second side of the second rib portion to secure the second implant to the second rib portion.

In Example 12, the subject matter of Example 11 optionally includes wherein the first body includes a slot and the second body includes a bar insertable into the slot to, together, define the reduction member, the bar translatable within the slot to guide movement of the first implant with respect to the second implant.

In Example 13, the subject matter of Example 12 optionally includes wherein the bar and the slot include a ratcheting interface.

In Example 14, the subject matter of any one or more of Examples 10-13 optionally include wherein the first body includes a bore and the first hook includes a rod insertable into the bore to secure the first hook to the first body.

In Example 15, the subject matter of Example 14 optionally includes wherein the rod includes a plurality of teeth engageable with a pawl of the bore to form a ratcheting interface between the rod and the first body.

In Example 16, the subject matter of any one or more of Examples 10-15 optionally include wherein the reduction member is a sliding bar and channel interface of the first implant and the second implant.

Example 17 is a method of repairing a fractured rib using a rib fixation system, the method comprising: securing a first implant to a first rib portion of a rib; securing a second implant to a second rib portion of the rib; connecting a reduction member to the first implant and the second implant; and operating the reduction member to move the second implant and the second rib portion toward the first implant and the first rib portion.

In Example 18, the subject matter of Example 17 optionally includes connecting a third implant to a third rib portion of the rib; and connecting the reduction member to the third implant.

In Example 19, the subject matter of Example 18 optionally includes operating the reduction member to move the third implant and the third rib portion toward the first implant, the second implant, the first rib portion, and the second rib portion.

In Example 20, the subject matter of any one or more of Examples 17-19 optionally include connecting the first implant to the second rib portion following operating the reduction member.

Example 21 is a rib implant system comprising: a first implant securable to a first rib portion of a rib; a second implant securable to a second rib portion of the rib; and a tether connected to the first implant and the second implant.

In Example 22, the subject matter of Example 21 optionally includes wherein the first implant includes a first retention passage extending through at least a portion of the first implant, wherein the second implant includes a second retention passage extending through at least a portion of the second implant, the first retention passage and the second retention passage to receive at least a portion of the tether therethrough to align the second implant with the first implant and to align the second rib portion with the first rib portion.

In Example 23, the subject matter of Example 22 optionally includes wherein the tether includes a head engageable with the first implant to limit movement of the tether with respect to the first implant.

In Example 24, the subject matter of Example 23 optionally includes wherein the tether includes a first portion securable to a second portion to adjust a length of the tether.

In Example 25, the subject matter of Example 24 optionally includes wherein the tether includes a lock operable to limit movement of the first portion of the tether with respect to the second portion.

In Example 26, the subject matter of Example 25 optionally includes wherein the lock is engageable with the second implant to limit movement of the tether with respect to the first implant and the second implant.

In Example 27, the subject matter of any one or more of Examples 21-26 optionally include a third implant securable to the first rib portion, the third implant movable along the tether with respect to the first implant and the second implant.

In Example 28, the subject matter of Example 2.7 optionally includes wherein the first implant includes a first retention passage extending through at least a portion of the first implant, wherein the second implant includes a second retention passage extending through at least a portion of the second implant, the first retention passage and the second retention passage to receive at least a portion of the tether therethrough to align the second implant with the first implant and to align the second rib portion with the first rib portion.

In Example 29, the subject matter of Example 28 optionally includes wherein the third implant includes a third retention passage extending through at least a portion of the third implant, the third retention passage configured to guide movement of the third implant along the tether.

In Example 30, the subject matter of Example 29 optionally includes wherein the tether is welded to the first implant and the second implant.

In Example 31, the subject matter of Example 30 optionally includes a fourth implant securable to the second rib portion, the fourth implant movable along the tether with respect to the first implant and the second implant.

In Example 32, the subject matter of any one or more of Examples 21-31 optionally include wherein the first implant includes a first fastener bore to receive a first fastener therethrough to secure the first implant to the first rib portion.

In Example 33, the subject matter of any one or more of Examples 21-32 optionally include wherein the tether is made of a malleable metallic material.

In Example 34, the system, device, or method of any one of or any combination of Examples 1-33 is optionally configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more,” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed, feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A rib implant system comprising:
a first implant securable to a first rib portion of a rib;
a second implant securable to a second rib portion of the rib; and
a reduction member connected to the first implant and the second implant, the reduction member operable to move the second implant and the second rib portion toward the first implant and the first rib portion.

2. The rib implant system of claim 1, wherein the first implant includes a first fastener bore to receive a first fastener therethrough to secure the first implant to the first rib portion.

3. The rib implant system of claim 2, wherein the second implant includes a second fastener bore to receive a second fastener therethrough to secure the second implant to the second rib portion, and includes an overlap fastener bore to receive a third fastener therethrough to secure the second implant to the first rib portion.

4. The rib implant system of claim 1, further comprising:
a third implant securable to a third portion of the rib, the third implant including a reduction passage extending through at least a portion of the third implant, the reduction passage to receive at least a portion of the reduction member therethrough to align the third implant with the first implant and the second implant and to align the third rib portion with the second rib portion and the first rib portion when the reduction member is adjusted from a first configuration to a second configuration.

5. The rib implant system of claim 4, wherein the first implant includes a first fastener bore to receive a first fastener therethrough to secure the first implant to the first rib portion, wherein the second implant includes a second fastener bore to receive a second fastener therethrough to secure the second implant to the second rib portion, and wherein the third implant includes a third fastener bore to receive a fastener therethrough to secure the third implant to the third rib portion.

6. The rib implant system of claim 4, wherein the third implant includes a bone spike engageable with the third rib portion to secure the third implant to the third rib portion.

7. The rib implant system of claim 4, wherein the reduction member is a tether secured to the first implant and engageable with the second implant, the tether extendable through the third implant, such that operation of the tether aligns the third implant with the first implant and the second implant to align the third rib portion with the first rib portion and the second rib portion.

8. The rib implant system of claim 7, wherein the second implant includes a second reduction passage extending through at least a portion of the second implant, the second reduction passage to receive at least a portion of the tether therethrough to align the second implant with the first implant and the third implant and to align the second rib portion with the third rib portion and the first rib portion when the reduction member is operated.

9. The rib implant system of claim 7, wherein the third implant includes a second reduction passage extending through at least a portion of the third implant, the second reduction passage to receive at least a portion of the tether therethrough to align the third implant with the first implant and the second implant and to align the third rib portion with the first rib portion and the second rib portion when the reduction member is operated.

10. The rib implant system of claim 1, wherein the first implant includes a first body engageable with a first side of the first rib portion and a first hook engageable with a second side of the first rib portion to secure the first implant to the first rib portion.

11. The rib implant system of claim 10, wherein the second implant includes a second body engageable with a first side of the second rib portion and a second hook engageable with a second side of the second rib portion to secure the second implant to the second rib portion.

12. The rib implant system of claim 11, wherein the first body includes a slot and the second body includes a bar insertable into the slot to, together, define the reduction member, the bar translatable within the slot to guide movement of the first implant with respect to the second implant.

13. The rib implant system of claim 12, wherein the bar and the slot include a ratcheting interface.

14. The rib implant system of claim 10, wherein the first body includes a bore and the first hook includes a rod insertable into the bore to secure the first hook to the first body.

15. The rib implant system of claim 14, wherein the rod includes a plurality of teeth engageable with a pawl of the bore to form a ratcheting interface between the rod and the first body.

16. The rib implant system of claim 10, wherein the reduction member is a sliding bar and channel interface of the first implant and the second implant.

17. A rib implant system comprising:
a first implant securable to a first rib portion of a rib;
a second implant securable to a second rib portion of the rib; and
a tether connected to the first implant and the second implant.

18. The rib implant system of claim 17, wherein the first implant includes a first retention passage extending through at least a portion of the first implant, wherein the second implant includes a second retention passage extending through at least a portion of the second implant, the first retention passage and the second retention passage to receive at least a portion of the tether therethrough to align the second implant with the first implant and to align the second rib portion with the first rib portion.

19. The rib implant system of claim 18, wherein the tether includes a head engageable with the first implant to limit movement of the tether with respect to the first implant.

20. The rib implant system of claim 19, wherein the tether includes a first portion securable to a second portion to adjust a length of the tether.
